(19) United States
(12) Patent Application Publication
(10) Pub. No.: US 2007/0135488 A1
(43) Pub. Date: Jun. 14, 2007

(54) Amlodipine nicotinate and process for the preparation thereof

(76) Inventors: You Sup Chung, Suwon-City (KR); Mun Choun Ha, Yongin-City (KR)

Correspondence Address: ROTHWELL, FIGG, ERNST & MANBECK, P.C., 1425 K STREET, N.W., SUITE 800, WASHINGTON, DC, 20005, US

(21) Appl. No.: 10/411,338

(22) Filed: Apr. 11, 2003

(30) Foreign Application Priority Data
Apr. 13, 2002 (KR) 2002-20268
Jan. 9, 2003 (KR) 2003-1259

(51) Int. Cl. A61K 31/444; C07D 401/02; C07D 213/79

(52) U.S. Cl. 514/332; 546/263

(57) ABSTRACT

The present invention provides a novel salt of amlodipine, i.e., a nicotinic acid salt of amlodipine, a process for preparing the same, and a pharmaceutical composition comprising the same as an active ingredient.

This application is based upon and claims priorities from Korean Patent Application Nos. 10-2002-20268 filed Apr. 13, 2002 and 10-2003-1259 filed Jan. 9, 2003, the contents of which are incorporated herein by reference.

### BACKGROUND OF THE INVENTION

1. Field of the Invention

The present invention relates to a novel salt of amlodipine, more specifically, to a nicotinic acid salt of amlodipine, a process for preparing the same, and a pharmaceutical composition comprising the same as an active ingredient.

2. Description of the Related Art

Amlodipine, with a chemical name of 3-ethyl 5-methyl 2-(2-aminoethoxymethyl)-4-(2-chlorophenyl)-1,4-dihydro-6-methylpyridine-3,5-dicarboxylate, is a potent and long-acting calcium channel blocker useful as an anti-ischaemic and anti-hypertensive agent.

Although amlodipine is effective as a free base form, in practice, it is administered in a form of a pharmaceutically acceptable acid addition salt. Such a pharmaceutically acceptable salt of amlodipine must satisfy the following four pharmaceutical criteria: (1) solubility; (2) stability; (3) non-hygroscopicity; (4) processability for tablet formulation.

Generally, a certain level of aqueous solubility is necessary for bioavailability. Usually, a solubility greater than 1 mg/ml at pH 1-7.5 is recommended although a higher solubility is required to formulate injections. In addition, salts which provide solutions having a pH close to a blood pH (pH 7.4) are preferred because they are readily biocompatible and can easily be buffered to a required pH range without altering their solubility.

A stability in a solid state is considered for tablets and capsules, while a stability in a solution is considered for an aqueous injection.

In order to provide stable formulations, it is desirable to use a non-hygroscopic salt. In a solid state having a high drug content, films with absorbed moisture can act as a vector for hydrolysis and chemical breakdown. The hygroscopic nature of a drug or its salt contributes to the generation of a free moisture which normally leads to unstable formulations.

As for processability, the compression properties and the ability not to stick or adhere to the tablet making machinery are to be considered. In high dose formulations, good compressibility is important to make elegant tablets. With lower dose tablets, the need for good compressibility may not be as vital due to the use of suitable diluting excipients called compression aids. Microcrystalline cellulose is a commonly used compression aid. However, regardless of the dose, the adhesion of a drug to the punches of a tablet machine is to be avoided. When drug accumulates on a surface of the punches, the tablet surface becomes pitted and therefore becomes undesirable. Also, such adhesion of drug on a machine requires a high ejection force to remove the tablet from the machine. In practice, it is possible to reduce the adhesion by wet-massing, careful selection of excipients and the use of a great amount of anti-adherents, e.g. magnesium stearate. However, by selecting a salt with good anti-adhesion properties, these problems are minimized.

EP 89,167 and U.S. Pat. No. 4,572,909 disclose various different pharmaceutically acceptable salt forms of amlodipine. In particular, pharmaceutically acceptable acid addition salts are disclosed, formed from acids which form non-toxic acid addition salts containing pharmaceutically acceptable anions, such as the hydrochloride, hydrobromide, sulfate, phosphate or acid phosphate, acetate, maleate, fumarate, lactate, tartrate, citrate and gluconate salts. Further, among them, maleate salt is disclosed as a preferable salt.

EP 244,944 and U.S. Pat. No. 4,879,303 disclose that benzene sulphonate salt of amlodipine (amlodipine besylate) has a number of advantageous physicochemical properties over the maleate salt thereof, such as good solubility, good stability, non-hygroscopicity, and processability for tablet formulation.

However, amlodipine besylate has a low photostability. Further, the pH of amlodipine besylate at saturation is not sufficiently close to the pH of a blood (pH 7.4±0.5).

### SUMMARY OF THE INVENTION

The present invention provides a novel amlodipine salt, i.e., amlodipine nicotinate, which has an improved photostability; a pH at saturation sufficiently close to the pH of a blood (pH 7.4±0.5); good physicochemical properties such as solubility, stability, non-hygroscopicity, and processability; and an enhanced pharmacological activity.

Further, the present invention provides a process for preparing the nicotinic acid salt of amlodipine and a pharmaceutical composition comprising amlodipine nicotinate.

In one aspect of the present invention, there is provided a nicotinic acid salt of amlodipine (i.e., amlodipine nicotinate).

In another aspect of the present invention, there is provided a process for preparing amlodipine nicotinate, which comprises reacting amlodipine with nicotinic acid in an organic solvent.

In still another aspect of the present invention, there is provided a process for preparing amlodipine nicotinate anhydrate, which comprises drying a hydrous form of amlodipine nicotinate.

In still another aspect of the present invention, there is provided a pharmaceutical composition for anti-ischaemia or anti-hypertension comprising a therapeutically effective amount of amlodipine nicotinate and a pharmaceutically acceptable carrier.

### BRIEF DESCRIPTION OF THE DRAWINGS

The above features and advantages of the present invention will become more apparent by describing in detail illustrative, non-limiting embodiments thereof with reference to the attached drawings, in which:

FIG. 1 shows a H-NMR chart of amlodipine nicotinate;

FIG. 2 shows an X-ray diffraction chart of amlodipine nicotinate;

FIG. 3 shows peak list data of the X-ray diffraction chart;

FIGS. 4A and 4B show H-NMR charts of amlodipine besylate before and after stability test, respectively;

FIGS. 5A and 5B show H-NMR charts of amlodipine nicotinate before and after stability test, respectively;

FIGS. 6A and 6B show H-NMR charts of amlodipine besylate before and after photostability test, respectively;

FIGS. 7A, 7B, and 7C show H-NMR charts of amlodipine besylate before hygroscopicity test, after hygroscopicity test, and after re-drying under a reduced pressure, respectively;

FIG. 8 is a graph illustrating the anti-hypertensive effects of amlodipine besylate on spontaneously hypertensive rats (Vehicle: ◯, Test Group 1 (1 mg/kg): ▴, Test Group 2 (3 mg/kg): ▾, and Test Group 3 (10 mg/kg): ▪);

FIG. 9 is a graph illustrating the anti-hypertensive effects of amlodipine nicotinate on spontaneously hypertensive rats (Vehicle: ◯, Test Group 4 (1 mg/kg): ▴, Test Group 5 (3 mg/kg): ▾, and Test Group 6 (10 mg/kg): ▪); and

FIG. 10 shows dose-response curves for the maximal changes of systolic blood pressure of amlodipine besylate and amlodipine nicotinate in spontaneously hypertensive rats (Amlodipine besylate: ◯ and Amlodipine nicotinate: ▴).

### DETAILED DESCRIPTION OF THE INVENTION

The nicotinic acid salt of amlodipine according to the present invention has a following chemical structure:

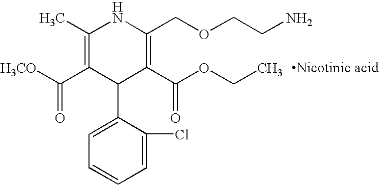

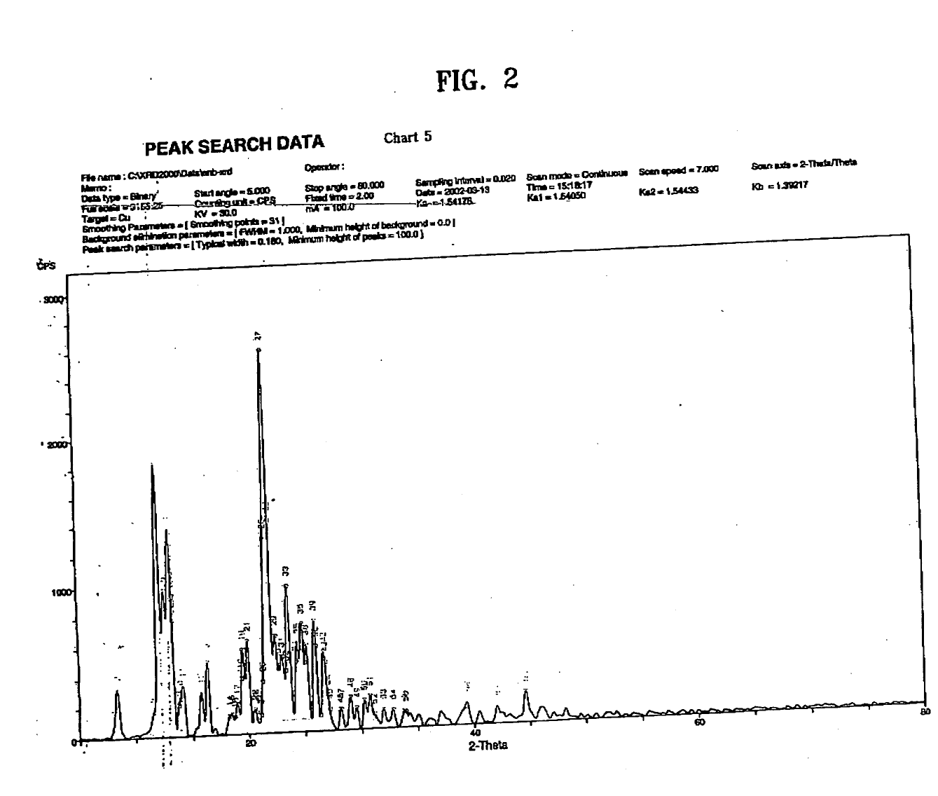

Amlodipine nicotinate of the present invention may be in an anhydrous form or a hydrous form. Preferably, amlodipine nicotinate is amlodipine nicotinate dihydrate (2H₂O), more preferably amlodipine nicotinate dihydrate having an X-ray diffraction pattern of FIG. 2.

Amlodipine nicotinate of the present invention has good physicochemical properties such as good solubility, good stability, non-hygroscopicity, and processability for tablet formulation, which is clear from various Examples to be described afterwards.

Further, amlodipine nicotinate of the present invention has a high photostability and a pH at saturation sufficiently close to that of human blood (pH 7.4), which allows it to be readily biocompatible and easily buffered to a required pH range without altering its solubility.

The present invention also includes, within its scope, a process for preparing amlodipine nicotinate. That is, the present invention provides a process for preparing amlodipine nicotinate, which comprises reacting amlodipine with nicotinic acid in an organic solvent.

In the process of the present invention, the organic solvent used includes any conventional solvent capable of dissolving both amlodipine and nicotinic acid, such as C₁-C₅ alkanol including methanol, ethanol, isopropanol etc. Further, the organic solvent used includes a conventional solvent containing water, e.g., 95% industrial methylated spirit, etc.

The process of the present invention may further comprise a re-crystallization step. Preferably, a mixed solvent of methanol and isopropanol or water and isopropanol is used. When a mixed solvent of methanol and isopropanol is used, methanol and isopropanol may be mixed in a ratio of about 1:9 to 2:8 by volume. When a mixed solvent of water and isopropanol is used, water and isopropanol may be mixed in a ratio of about 3:97 to 5:95 by volume. However, the mixing ratios of the solvents may vary according to a person skilled in the art.

Further, the present invention provides a process for preparing amlodipine nicotinate anhydrate, which comprises drying a hydrous form of amlodipine nicotinate. The drying step may be performed under a reduced pressure and at 115-125° C.

The present invention includes, within its scope, a pharmaceutical composition for anti-ischaemia or anti-hypertension comprising a therapeutically effective amount of the amlodipine nicotinate and a pharmaceutically acceptable carrier.

The pharmaceutical composition of the present invention may be administered orally or parenterally. The pharmaceutical composition for oral administration may be in various forms such as tablets, capsules, granules, and solutions, which may further contain conventional additives such as a diluent, disintegrant, lubricant and the like. The composition for parenteral administration (e.g., injection) may be an isotonic solution, and may be sterilized and/or may contain a conventional adjuvant such as a preservative, stabilizer and the like.

The pharmaceutical composition of the present invention may be administered for the treatment of ischaemia or hypertension in a dosage of about 2-50 mg/day for an average adult of about 70 kg weight, although the dosage may vary in accordance with the kind and severity of a disease to be treated. Thus, for a typical adult patient, individual tablets or capsules may contain about 1 to 10 mg of amlodipine nicotinate, in a suitable pharmaceutically acceptable carrier. Dosages for intravenous administration would be about 1 to 10 mg per single dose as necessary.

Although the present invention herein may be more detailed explained by reference to the following Examples. The following Examples are not intended to limit the scope of the present invention.

### EXAMPLE 1

### Preparation of amlodipine nicotinate dihydrate

The solution of amlodipine (10.0 g, 24.45 mmole) in 95% industrial methylated spirit (40.0 ml) was added to the slurry of nicotinic acid (3.0 g, 24.37 mmole) in 95% industrial methylated spirit (10 ml). The solution was slowly heated and then refluxed for 3 hours. The reaction mixture was cooled to 5° C. to form amlodipine nicotinate hydrate, which was then filtered and washed with industrial isopropanol (20.0 ml).

The resulting salt was heated and dissolved in the mixed solvent (40 ml) of 95% methanol and isopropanol (1:9 by volume). The resulting solution was slowly stirred at a room temperature and cooled to 5° C. to produce a precipitate, which was then filtered, washed with isopropanol (20.0 ml), and dried under a reduced pressure and at 80° C. for 5 hours to give 11.0-11.3 g of amlodipine nicotinate.

Yield: 79.3-81.4%

Melting Point: 174-176° C.

H-NMR (CDCl₃) 9.17(s, 1H), 8.60(d, 1H), 8.19(d, 1H), 7.91(s, 1H), 6.99-7.30(m, 5H), 5.31(s, 1H), 4.69(gq, 2H), 4.00(m, 2H), 3.76(bs, 2H), 3.55(s, 3H), 3.18(bs, 2H), 2.21(s, 3H), 1.15(t, 3H).

200 mg of amlodipine nicotinate obtained in the above process was dried at 120° C. and under a reduced pressure of lower than 5 mmHg for 5 hours and afterwards, the loss on dry (LOD) thereof was measured. As a result, the obtained amlodipine nicotinate in Example 1 was in the form of amlodipine nicotinate dihydrate.

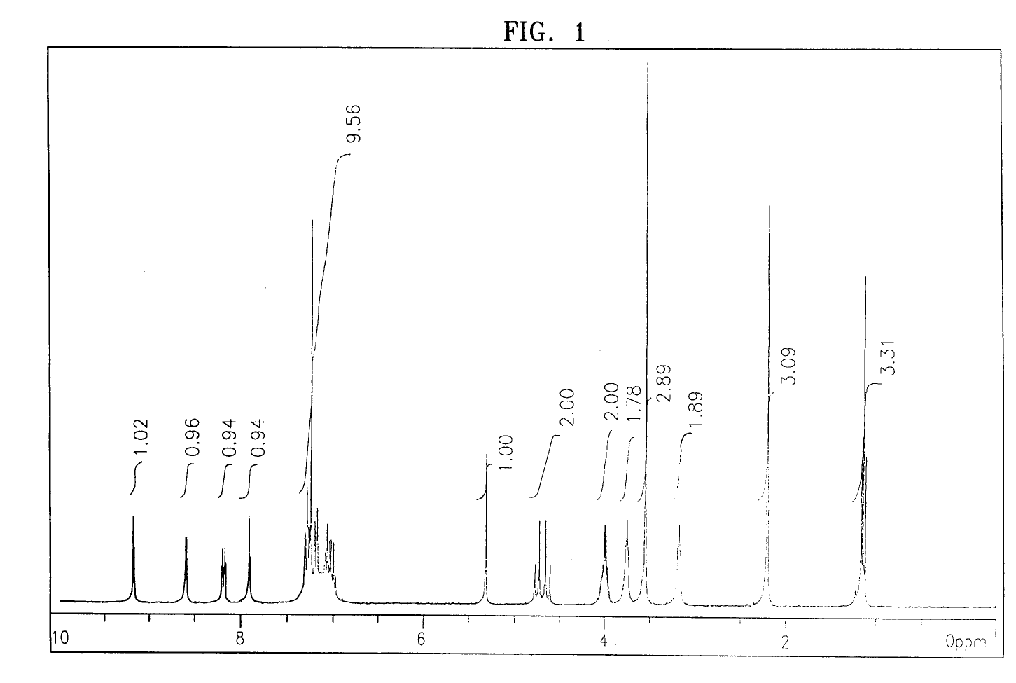

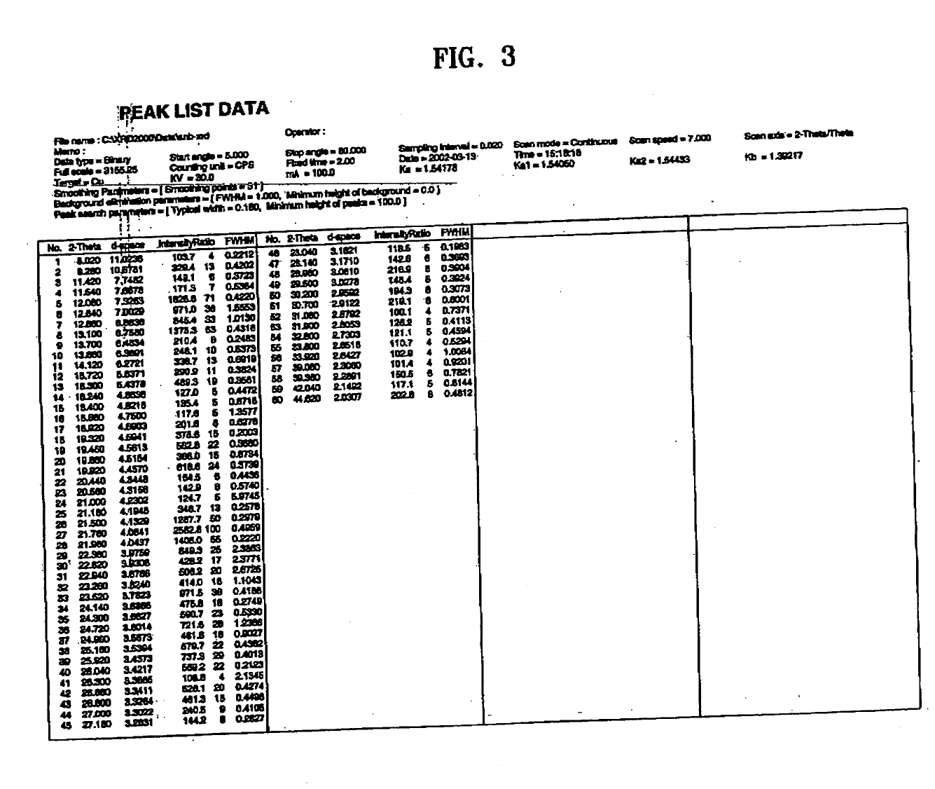

The H-NMR chart of the product obtained in the above process is shown in FIG. 1. Further, the X-ray diffraction of the product obtained in the above process, which was measured with Rigaku Rotaflex 12Kw XRD-2000, is shown in FIG. 2 and the peak list data thereof are shown in FIG. 3.

### EXAMPLE 2

### Preparation of amlodipine nicotinate dihydrate

The procedure of Example 1 was repeated, except for using the mixed solvent (40 ml) of water and isopropanol (5:95 by volume) in place of the mixed solvent (40 ml) of 95% methanol and isopropanol (1:9 by volume), to obtain 11.2-11.4 g of amlodipine nicotinate dihydrate.

### EXAMPLE 3

### Preparation of amlodipine nicotinate anhydrate

Amlodipine nicotinate dihydrate obtained in Example 1 was dried under a reduced pressure and at 115-125° C. for 5 hours to give amlodipine nicotinate anhydrate.

Melting Point: 176-177° C.

| | | | |
|---|---|---|---|
| Calc. | C; 58.70 | H; 5.68 | N: 7.90 |
| Found | C; 58.62 | H; 5.65 | N: 7.94 |

### TEST EXAMPLE 1

### Solubility Measurement

Water solubilities were measured for amlodipine besylate prepared in accordance with U.S. Pat. No. 4,879,303 and amlodipine nicotinate obtained in Example 1. By using 50 ml of distilled water and ultrasonic waves, maximal solubilized amounts were measured at a room temperature and pHs at saturation were measured using Fischer Scientific Accument (PH meter 15). The results are shown in Table 1.

TABLE 1

| Salt | Solubility (mg/ml) | pH at saturation |
|---|---|---|
| Amlodipine besylate | 4.6 | 6.6 |
| Amlodipine nicotinate | 6.8 | 7.2 |

The results in Table 1 indicate that amlodipine nicotinate exhibits higher solubility than amlodipine besylate. Further, the pH at saturation of amlodipine nicotinate is closer to that of a blood, compared with amlodipine besylate.

### TEST EXAMPLE 2

### Stability Test

(1) Chemical Stability (Stability Test Under Stress Condition)

In order to assess the chemical stability, amlodipine besylate and amlodipine nicotinate were blended in a powder vehicle and formed into tablets. The vehicle for tablets comprised microcrystalline cellulose and anhydrous dibasic calcium phosphate in 50:50. The tablets were then stored in sealed vials at 50° C. and at 60% relative humidity for three weeks. Afterwards, the drug and any breakdown products thereof were extracted with a mixed solvent of methanol and chloroform (50:50) and separated on silica TLC plates using a developing solvent (CHCl₃:MeOH:MeOH:acetic acid:H₂O=40:10:5:2 by volume).

There was no breakdown product for amlodipine nicotinate. The Rf values for breakdown products of amlodipine besylate were measured (the Rf value for breakdown products of amlodipine besylate was 0.38) and peaks on the NMR thereof were observed, using 300 MHz FT-NMR Spectrometer (JEOL JNM-LA300).

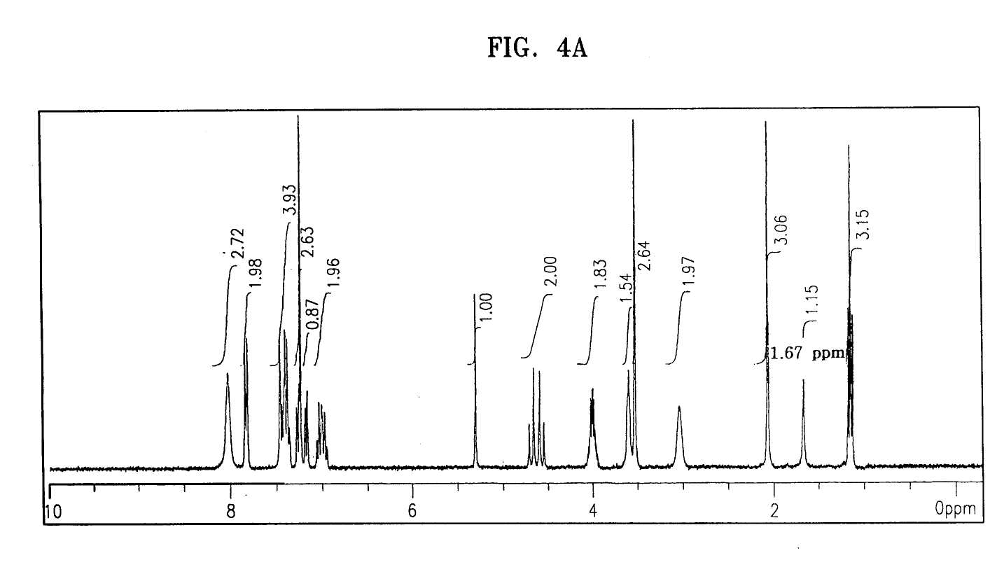

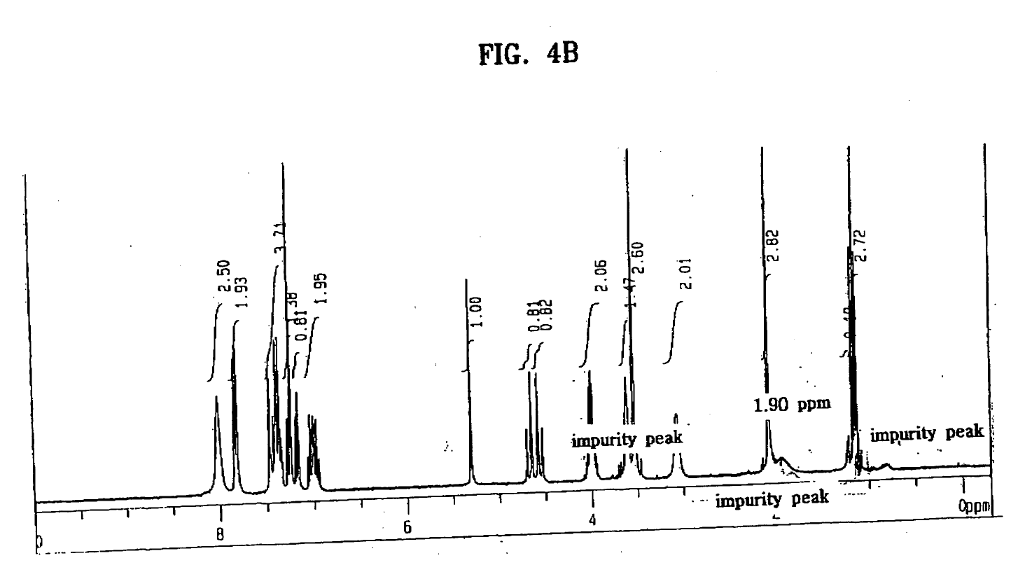

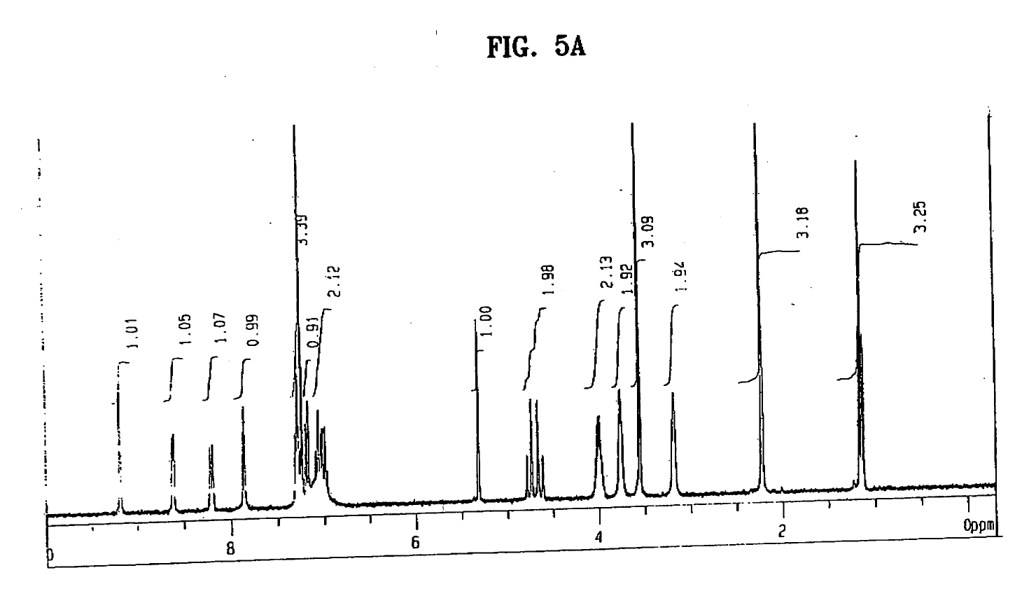

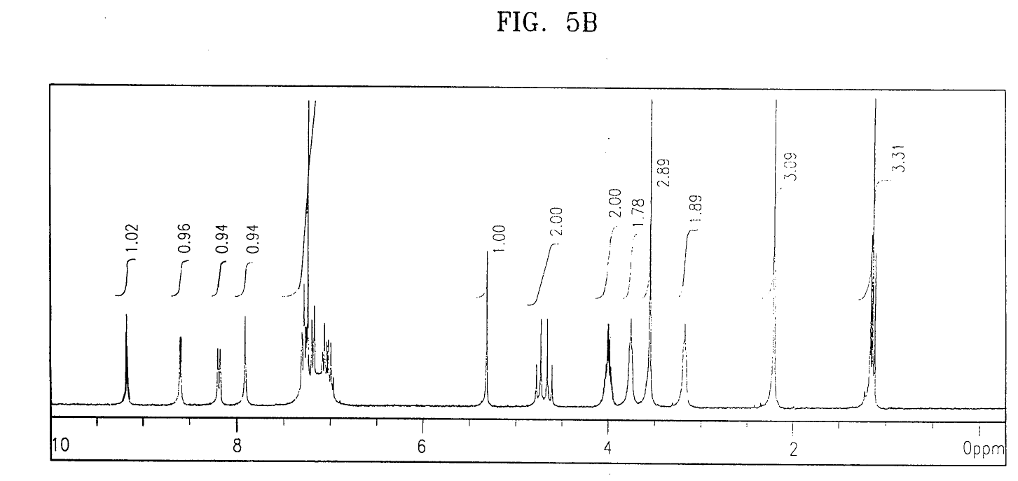

The results of the NMR measurement are shown in FIGS. 4 and 5. FIGS. 4A and 4B show H-NMR charts of amlodipine besylate before and after the stability test, respectively, and FIGS. 5A and 5B show H-NMR charts of amlodipine nicotinate before and after the stability test, respectively. The peaks on NMR of amlodipine besylate are as follows:

| | |
|---|---|
| Before stability test: | a peak at 1.67 ppm (bs, —NH₂) |
| After stability test: | no peak at 1.67 ppm and a broad peak around 1.90 |
| | ppm and impurity peaks |

(2) Photostability

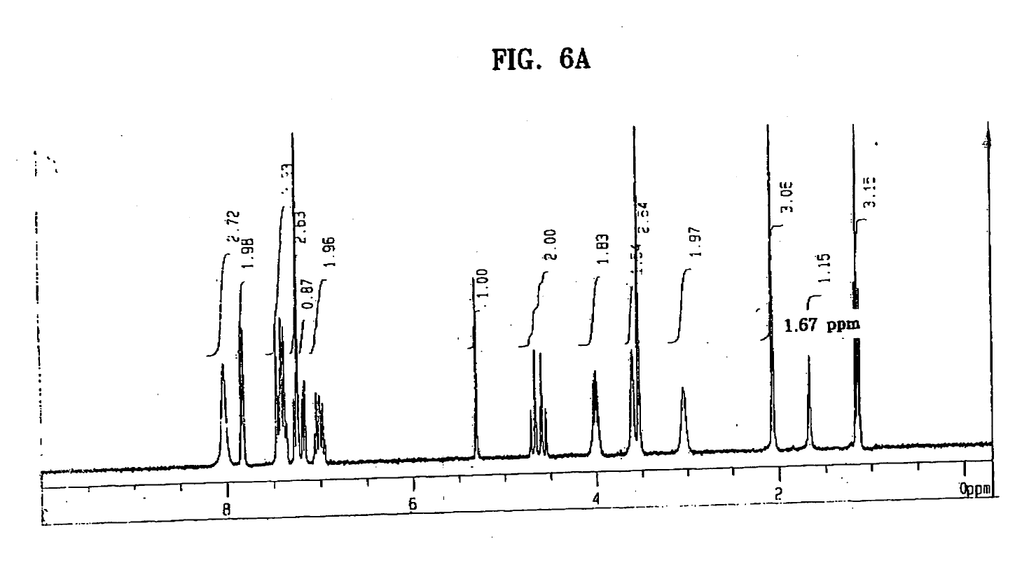

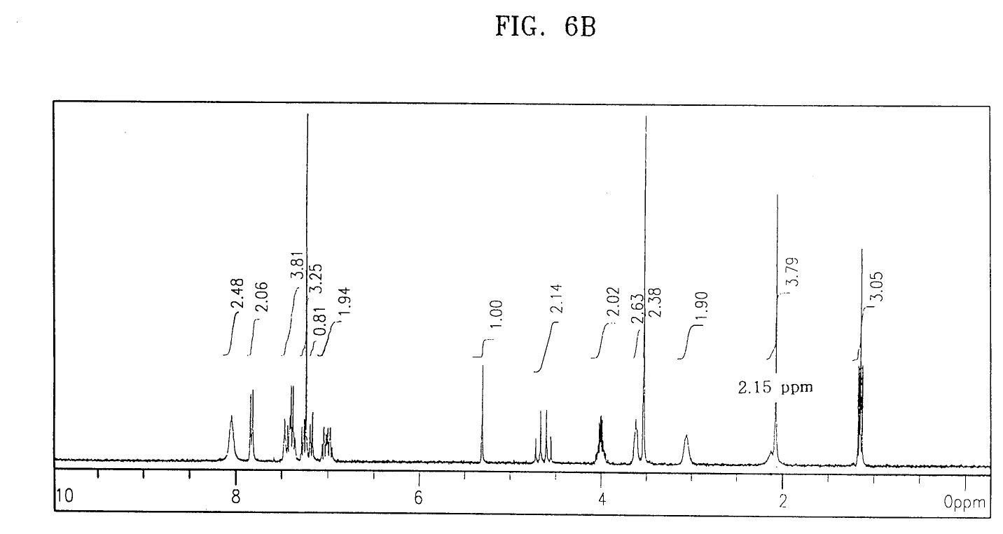

1.0 g of amlodipine besylate prepared in accordance with U.S. Pat. No. 4,879,303 and 1.0 g of amlodipine nicotinate dihydrate obtained in Example 1, which were placed in glass schales (100×20 mm), were exposed at 25-30° C. for 2 weeks under an incandescent lamp (220V, 100 W) that was placed at 30 cm above the samples. As a result, amlodipine besylate was discolored to yellow, while there was no color change in amlodipine nicotinate dihydrate. FIGS. 6A and 6B show H-NMR charts of amlodipine besylate before and after the photostability test, respectively. The peaks on NMR of amlodipine besylate are as follows:

| | |
|---|---|
| Before stability test: | a peak at 1.67 ppm (bs, —NH₂) |
| After stability test: | no peak at 1.67 ppm and broad peak at 2.15 ppm |

### TEST EXAMPLE 3

### Hygroscopicity Test

1.0 g of amlodipine besylate prepared in accordance with U.S. Pat. No. 4,879,303 and 1.0 g of amlodipine nicotinate dihydrate obtained in Example 1 were exposed to 60% relative humidity at 50° C. for 14 days. As a result, amlodipine nicotinate dihydrate remained intact and amlodipine besylate remained anhydrous.

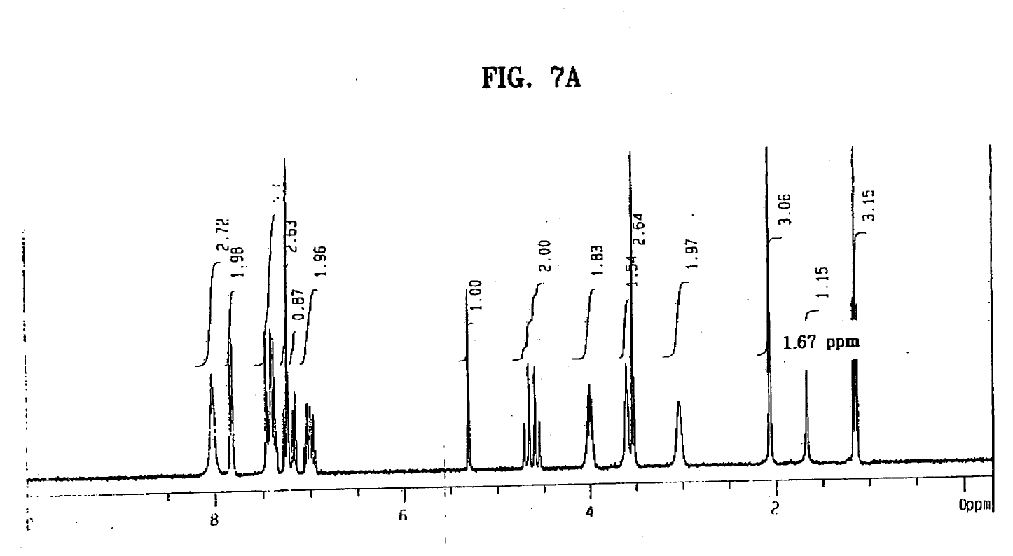

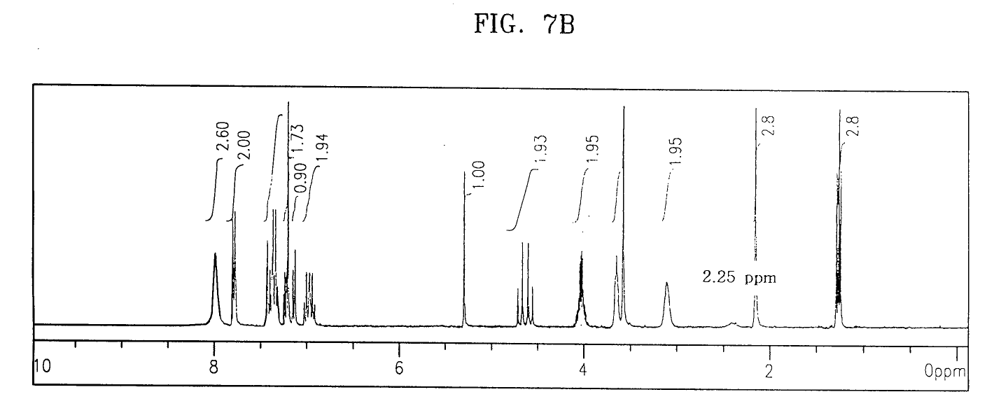

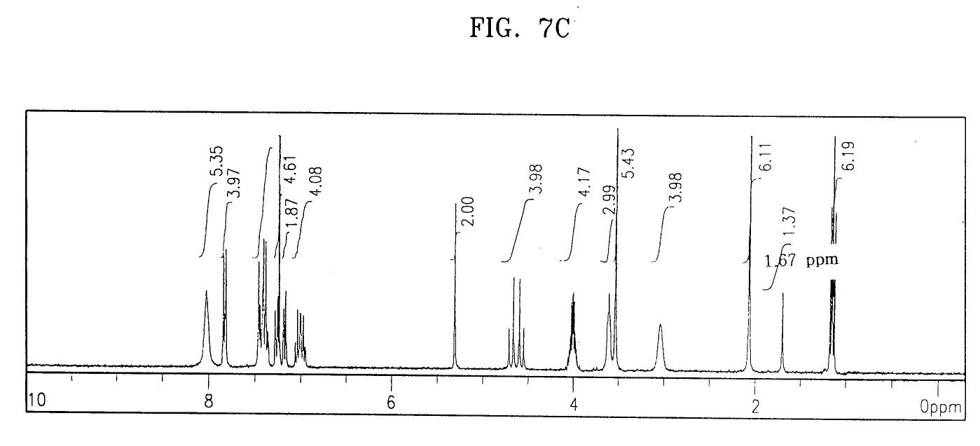

Meanwhile, when H-NMRs of amlodipine besylate were measured before and after the test, the —NH₂ peak was shifted from around 1.67 ppm to around 2.25 ppm and the height of the peak decreased (FIGS. 7A and 7B). Further, when the resulting amlodipine besylate was dried under a reduced pressure at 120° C. for 5 hours and the H-NMR was re-measured, the —NH₂ peak was re-observed at around 1.67 ppm, the position before the hygroscopicity test (FIG. 7C). Therefore, a stability problem of amlodipine besylate can be inferred aside from the hygroscopicity test result.

### TEST EXAMPLE 4

### Processability Test

Using a conventional tablet making machinery, fifty tablets containing calcium sulphate dihydrate, microcrystalline cellulose and amlodipine nicotinate dihydrate (47.5:47.5:5) were produced. The material adhering to the tablet punch was then extracted using methanol and the amount was measured spectrometrically. This procedure was then repeated for runs of 100, 150, 220, 250 and 300 tablets. After each run the amount of material adhering to the tablet punch was measured in the same process as above. The values were plotted and an average value was calculated from the slope of the line produced. The same procedure was then repeated for amlodipine besylate. The amount of amlodipine measured as adhering to the tablet punch is shown in Table 2 for amlodipine nicotinate dihydrate with relative to amlodipine besylate.

TABLE 2

Stickiness

| Salt | Amlodipine(μg)/cm² (tablet) | Relative to besylate |
|---|---|---|
| Nicotinate | 0.44 | 36.7% |
| Besylate | 1.20 | 100.0% |

The results in Table 2 indicate amlodipine nicotinate has superior anti-adhesion properties to amlodipine besylate.

As clear from Test Examples 1 to 4 above, the nicotinic acid salt of amlodipine shows improved physicochemical properties such as solubility, stability, non-hygroscopicity and processability, which makes it suitable for the preparation of pharmaceutical formulations of amlodipine.

### TEST EXAMPLE 5

### Comparison of Pharmacological Effects Induced by amlodipine besylate and amlodipine nicotinate

Cardiovascular effects, i.e., in vivo anti-hypertensive activities, were measured for amlodipine besylate prepared in accordance with U.S. Pat. No. 4,879,303 and amlodipine nicotinate prepared in Example 1, using spontaneously hypertensive rats (SHRs), by Korea Research Institute of Chemical Technology (Screening Center, #100, Jang-dong, Yuseong-gu, Daejeon).

(1) Animal Used

Male SHRs (Charles Rever Co., Japan) aged 13-14 weeks were used. Before evaluation, the SHRs were accustomed in a clean breeding chamber under conditions of a temperature of 22.5±1° C., a relative humidity of 55±5% and a lighting time of 12 hour intervals.

The SHRs having a systolic blood pressure over 170 mmHg were divided into 7 groups, i.e., Test Groups 1 to 3 (for amlodipine besylate), Test Groups 4 to 6 (for amlodipine nicotinate) and a Control Group. Each Test Group and Control Group consisted of 6-8 SHRs (n=6-8).

(2) Preparation and Administration

The test compounds were dissolved in distilled water to prepare test solutions immediately prior to administration. The test solutions of amlodipine besylate and amlodipine nicotinate were prepared by dissolving 1, 3, and 10 mg/kg in distilled water (0.5 ml/100 g rat), respectively, and then administered orally to each Test Group. The vehicle (distilled water) was administered to Control Group.

(3) Measurement

The systolic blood pressure was measured with Multichannel 8000 (TSE Co., Germany), using a tail-cuff method. That is, the systolic blood pressures of a tail artery of each rat were measured before the administration of the test solutions and after 2, 4, 6, 8, 10, and 24 hours from the administration thereof. In order to facilitate the measurement of blood pressures, the test animals of each Group underwent warming at 37° C. for about 10 minutes before the measurements.

(4) Statistical Processing Method

The results of the foregoing test were expressed by a mean percentage and standard error (mean %±S.E.M.). Statistical analysis of the test results were conducted by an unpaired t-test and ANOVA (one-way analysis of variance) with Sigma Stat program (Jandel Co., USA). The secondary evaluations were conducted by a Dunnett's multiple comparison test.

(5) Results

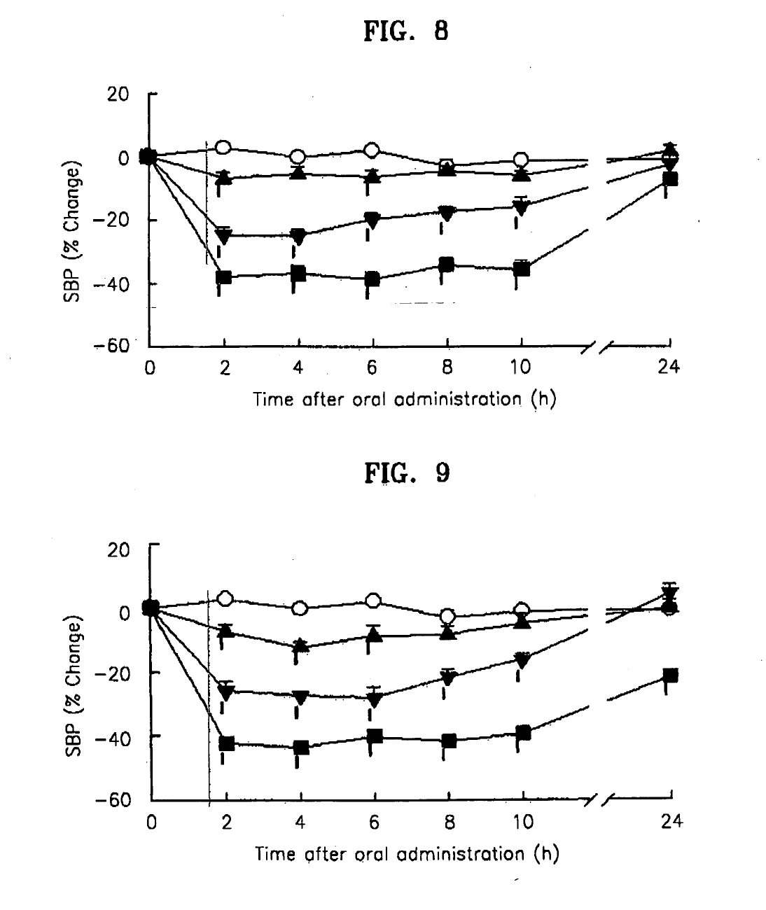

The test results are shown in FIGS. 8 to 10 and Tables 3 & 4. Both amlodipine besylate (FIG. 8 and Table 3) and amlodipine nicotinate (FIG. 9 and Table 3) dose-dependently reduced blood pressures. All Test Groups showed similar hypotensive (blood pressure falling) profiles. Substantial anti-hypertensive effects started to appear after 2 hours from the administrations and the maximal effects were displayed between 2 hours and 6 hours. The anti-hypertensive effects were maintained for over 10 hours. In Test Groups to which the doses of 10 mg/kg were administered (Test Groups 3 and 6), substantial anti-hypertensive effects were maintained even after 24 hours from administration.

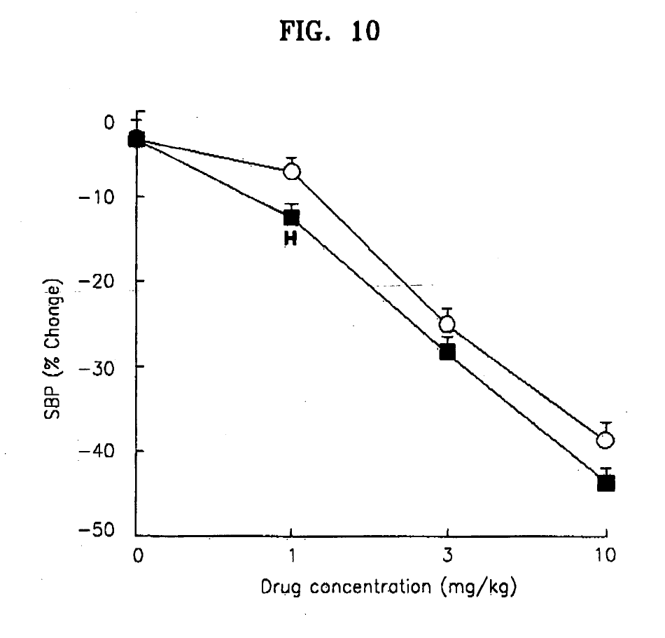

The maximal anti-hypertensive effects of each Test Group are shown in Table 3 and FIG. 10.

TABLE 3
Maximal anti-hypertensive effects of each Test Group

| Dose | Besylate salt | Nicotinate salt | Intensity |
|---|---|---|---|
| 1 mg/kg (Group 1 & 4) | −7.0 ± 1.66 | −10.20 ± 2.71 | 1.46 |
| 3 mg/kg (Group 2 & 5) | −25.0 ± 1.98 | −26.8 ± 3.22 | 1.07 |
| 10 mg/kg (Group 3 & 6) | −38.7 ± 2.18 | −40.9 ± 2.08 | 1.06 |

In Table 3, the intensity is the percentage of the maximal effect of amlodipine nicotinate to the maximal effect of amlodipine besylate.

As shown in Table 3 and FIG. 10, substantial difference was shown in the Test Groups (Groups 1 & 4) to which the doses of 1 mg/kg were administered (p<0.05 vs. amlodipine besylate). The amlodipine nicotinate showed anti-hypertensive activity about 1.46 times higher than amlodipine besylate at 1 mg/kg dose.

The ED₂₀ values (the amount necessary for 20% decrease in the blood pressure) of amlodipine besylate and amlodipine nicotinate were 2.48±0.46 mg/kg and 2.19±0.57 mg/kg, respectively, as shown in Table 4.

TABLE 4
ED₂₀ values

| | Concentration (mg/kg) | Intensity |
|---|---|---|
| Amlodipine besylate | 2.48 ± 0.46 | 1.00 |
| Amlodipine nicotinate | 2.19 ± 0.57 | 1.13 |

In Table 4, the intensity is the reverse percentage of ED₂₀ value of amlodipine nicotinate to ED₂₀ value of amlodipine besylate.

As shown in Table 4, amlodipine nicotinate showed anti-hypertensive activity about 1.13 times higher than amlodipine besylate.

While this invention has been particularly shown and described with reference to preferred embodiments thereof, it will be understood by those skilled in the art that various changes in form and details may be made therein without departing from the spirit and scope of the invention as defined by the appended claims.

1. An amlodipine nicotinate in a hydrous form.

2-3. (canceled)

4. The amlodipine nicotinate of claim 1, wherein the amlodipine nicotinate is in a dihydrate form.

5. The amlodipine nicotinate of claim 4, wherein the amlodipine nicotinate in a dihydrate form has an X-ray diffraction pattern as shown in FIG. 2.

6. A process for preparing amlodipine nicotinate in a hydrous form, which comprises reacting amlodipine with nicotinic acid in an organic solvent.

7. The process of claim 6, further comprising a re-crystallization step using a mixed solvent of methanol and isopropanol or water and isopropanol.

8. (canceled)

9. A pharmaceutical composition for anti-ischaemia or anti-hypertension comprising a therapeutically effective amount of the amlodipine nicotinate according to claim 1 and a pharmaceutically acceptable carrier.
